(19) United States
(12) Patent Application Publication
(10) Pub. No.: US 2008/0060153 A1
(43) Pub. Date: Mar. 13, 2008

(54) SELF-SANITIZING TOOTHBRUSH

(76) Inventor: John M. Jansheski, Maryville, TN (US)

Correspondence Address: LUEDEKA, NEELY & GRAHAM, P.C., P O BOX 1871, KNOXVILLE, TN, 37901, US

(21) Appl. No.: 11/530,057

(22) Filed: Sep. 8, 2006

(51) Int. Cl. A46B 9/04

(52) U.S. Cl. 15/105; 15/167.1

(57) ABSTRACT

A self-contained sanitizing toothbrush having a one-piece body of molded plastic construction having a handle portion and a head portion, bristles extending outwardly from the head portion of the body, one or more sources of ultraviolet light located on within the head portion of the body and configured to direct ultraviolet light onto the bristles to sanitize the bristles between uses of the toothbrush, and a circuit contained within the body configured to be activated by a user after use of the toothbrush. The circuit operates to illuminate the light sources for a predetermined period of time sufficient for sanitization purposes and then turn the light sources off.

### FIELD

This invention relates to the field of toothbrushes. More particularly, this invention relates to a toothbrush which includes apparatus internal to the toothbrush for sanitizing the toothbrush between uses.

### BACKGROUND

Regular sanitization of a toothbrush is desirable to kill harmful germs and bacteria often found on toothbrushes, including streptococcus, E. coli and salmonella. Ultraviolet light sanitizers are known for this purpose, but, are provided as separate electrical devices configured to hold or store one or more toothbrushes and expose the toothbrushes to ultraviolet light. Such devices typically require counter space and an electrical outlet. Such devices are inconvenient for travel. What is desired is a toothbrush which is self-contained and includes apparatus internal to the toothbrush for sanitizing the toothbrush between uses.

### SUMMARY

The above and other needs are met by a self-contained sanitizing toothbrush.

In a preferred embodiment, the toothbrush includes a one-piece body of molded plastic construction having a handle portion and a head portion, bristles extending outwardly from the head portion of the body, one or more sources of ultraviolet light located on within the head portion of the body and configured to direct ultraviolet light onto the bristles to sanitize the bristles between uses of the toothbrush, and a circuit contained within the body configured to be activated by a user after use of the toothbrush. The circuit operates to illuminate the light sources for a predetermined period of time sufficient for sanitization purposes and then turn the light sources off.

### BRIEF DESCRIPTION OF THE DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 is a perspective view of a self-sanitizing toothbrush according to an embodiment of the disclosure.

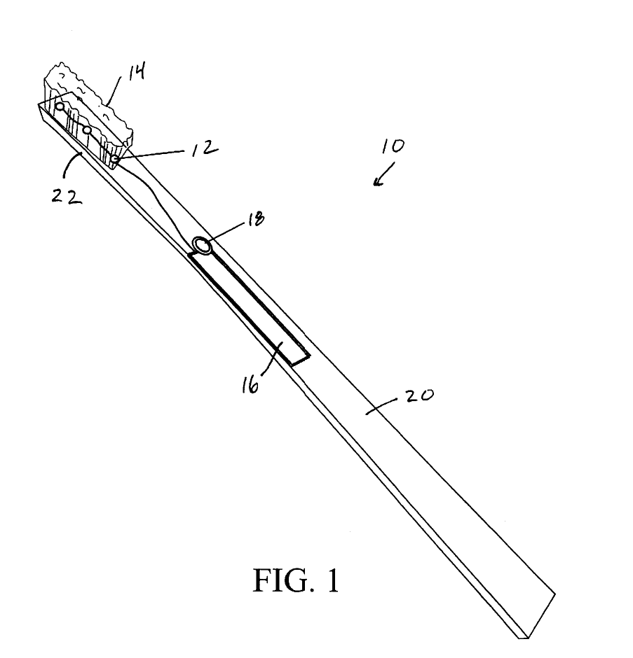

FIG. 2 is a schematic of an electrical circuit used in the toothbrush of FIG. 1.

FIG. 3 is a perspective view of a self-sanitizing toothbrush according to an alternate embodiment of the disclosure.

### DETAILED DESCRIPTION

With reference to the drawings, the disclosure relates to a toothbrush 10 which includes one or more ultraviolet light sources 12 configured to direct ultraviolet light to bristles 14 of the toothbrush, to sanitize the bristles 14 between uses. An associated electrical circuit 16 operated by a switch 18 enables a user to selectively activate the light sources 12.

The toothbrush 10 includes a one-piece body of molded plastic construction and may be constructed by molding a handle portion 20 thereof around the circuit 16, leaving the switch 18 exposed, and a head portion 22 thereof around bases of the bristles 14 and the light sources 12 and associated circuit components, leaving the light sources partially exposed to emit light in the direction of the bristles 14. The bristles 14 are made of nylon or other conventional toothbrush bristle material.

The ultraviolet light sources 12 are preferably light emitting diodes capable of producing ultraviolet light in the wavelength range of from about 100 nanometers to about 280 nanometers, known as UV-C. UV-C light is germicidal in that it damages the nucleic acid of microorganisms such as bacteria, viruses and other pathogens to destroy their ability to multiply and cause disease.

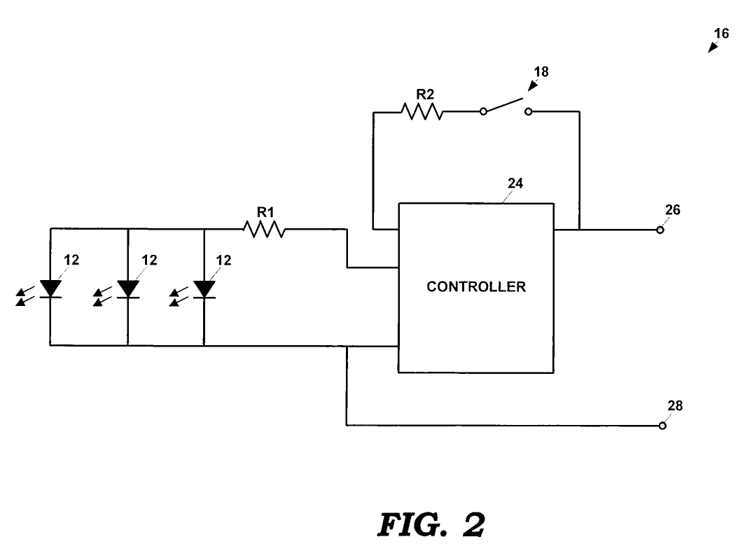

With reference to FIG. 2, there is shown a schematic of a preferred embodiment of the electrical circuit 16 for controlling activation of the light sources 12. In this regard, the circuit 16 is desirably configured such that a user, after use of the toothbrush 10 to brush teeth, may press the switch 18 to illuminate the light sources 12. The circuit 16 is configured to illuminate the light sources 12 for a predetermined period of time sufficient for sanitization purposes, such as five minutes, and then turn the sources 12 off.

The circuit 16 may include a microprocessor controller 24 that enables use of the switch 18. The controller 24 activates the light sources 12 when the switch 18 is pressed and automatically shuts off the light sources 12 after a predetermined time to conserve battery power. Positive and negative battery terminals 26 and 28 are provided for connection of one or more small batteries also located within the handle 20. In a preferred embodiment of the circuit 16, the resistor R1 has a value of 50 KΩ, and the resistor R2 has a value of 50 Ω.

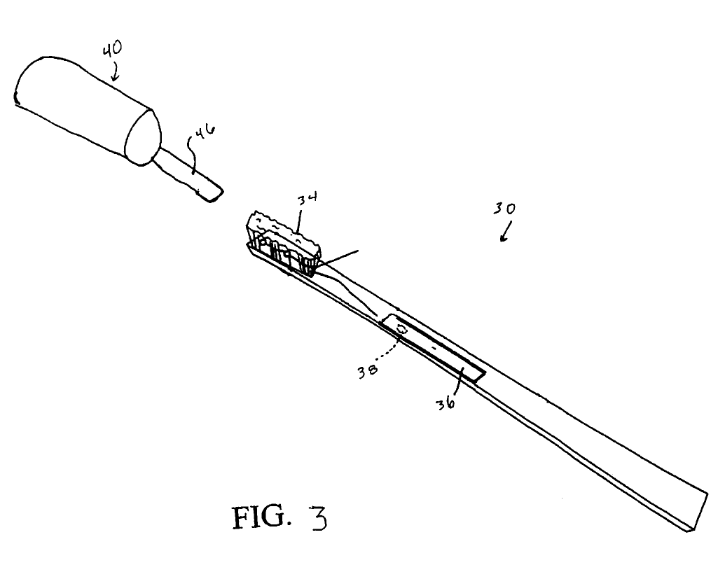

With reference to FIG. 3, there is shown an alternate embodiment of a toothbrush 30 which includes one or more ultraviolet light sources 32 configured to direct ultraviolet light to bristles 34 of the toothbrush, to sanitize the bristles 34 between uses. An associated electrical circuit 36 is operated by a switch 38 which is activated by installation of a cap 40.

The circuit 36 and switch 38 are substantially similar to the circuit 16 and switch 18 described previously. However, the switch 38 is preferably located adjacent an underside of handle 42 of the toothbrush 30 (opposite the bristles 34) and the switch 38 is configured as a press-switch so as to only enable operation of the light sources 32 when the cap 40 is installed.

For example, the cap 40 is preferably configured to enclose head 44 of the brush 30 including the bristles 34, so as to shield locations exterior to the cap from the light from the light sources 32. The cap 40 further includes an extension 46 configured to engage the switch 38 when the cap 40 is installed. Thus, when the switch 38 is depressed by installation of the cap 40, the circuit 36 is activated to turn the light sources 32 on for a predetermined period of time sufficient for sanitization purposes, such as five minutes, and then turn the sources 12 off. In the event the cap 40 is removed such that the switch 38 is disengaged, the circuit 36 will be in an open state and the light sources 32 will not operate. Thus, since the light sources 32 are only operable when the cap 40 is installed, a user is shielded from exposure to light from the light sources 32.

The foregoing description of preferred embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide the best illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A self-contained sanitizing toothbrush, comprising, a one-piece body of molded plastic construction having a handle portion and a head portion, bristles extending outwardly from the head portion of the body, one or more sources of ultraviolet light located on within the head portion of the body and configured to direct ultraviolet light onto the bristles to sanitize the bristles between uses of the toothbrush, and a circuit contained within the body configured to be activated by a user after use of the toothbrush, wherein when activated the circuit operates to illuminate the light sources for a predetermined period of time sufficient for sanitization purposes and then turn the light sources off.

2. The toothbrush of claim 1, wherein the light sources emit UV-C light having a wavelength of from about 100 to about 280 nanometers.

3. The toothbrush of claim 1, wherein the circuit includes a switch located on the handle portion.

4. The toothbrush of claim 1, further comprising a cap configured to substantially enclose the bristles to shield locations exterior to the cap from the light from the light sources and engage the switch such that the sources of ultraviolet light are operable only when the cap is installed.
